SEQUENCE LISTING (14 sequences)

SEQ ID NO: 1 - DNA, 32 bp, Artificial Sequence
  Description of Artificial Sequence: Synthetic primer
taagggtttttaagttgagtttatattttagt

SEQ ID NO: 2 - DNA, 28 bp, Artificial Sequence
  Description of Artificial Sequence: Synthetic primer
ctaatcccraactaaacaaacraactaa

SEQ ID NO: 3 - DNA, 28 bp, Artificial Sequence
  Description of Artificial Sequence: Synthetic primer
gtatgggtatygagtattgttaggtttt

SEQ ID NO: 4 - DNA, 29 bp, Artificial Sequence
  Description of Artificial Sequence: Synthetic primer
aaccctcacataatctaatctctaaataa

SEQ ID NO: 5 - DNA, 28 bp, Artificial Sequence
  Description of Artificial Sequence: Synthetic primer
cactttactcaaaaccctctttaaaaaa

SEQ ID NO: 6 - DNA, 29 bp, Artificial Sequence
  Description of Artificial Sequence: Synthetic primer
gtttttttttggygagygtaattttattt

SEQ ID NO: 7 - DNA, 27 bp, Artificial Sequence
  Description of Artificial Sequence: Synthetic primer
aaacccctacaacaataaacaaaaaaa

SEQ ID NO: 8 - DNA, 26 bp, Artificial Sequence
  Description of Artificial Sequence: Synthetic primer
ggggaagtattaagtttattcgtttt

SEQ ID NO: 9 - DNA, 800 bp, Homo sapiens
cgcccgcccacttcgagcactccacgttcctcgtctcccactctggccggtcagcttcagctctggctcttgctgagcgtggagggcgcaggggcaggatccggcgcccaccgctggggcgcatgcacctcctcgcctcctcgcagggcggtggggcgcagcggttctatccccctccccgaggcggggaagaaggtctggaaagaaacgtgggttcgaggcggagaggaaaagcggacccacctgccaggctgcgcggggaggctggtcccgggctgggcaggcgggctggcctcgcgccctcgaggcacccggcggcgctggctgtgcggaggggcgccggcgcggccgtatttgtacccgcgggccctcacatggtctgatctctagatagccgccgccaaagagctcttgaagaatttttgcgtcactttgaggcgaataaacttaatgcttccccgcggccgcggctccgcgctcccgctggatggggttgcgctcgccagggaggggccgcgctacggggcggggtgcgcgcccgaccccagagccaggaggggagggacccccgacacacacacacgctcgcagggaggagcggagcgcggagcagccggcagggcagggccgagcgaggaatcctgactttcctgctctttaactttgcgggaggggggaggctgcacaaaggagcaggtgtgcgcctcccctgccgccccgcgcccacaggacggcacacaggggtctgctcgtgccgctttctcttgacctcggacacctccccatcgtccccatcccga

SEQ ID NO: 10 - DNA, 83 bp, Homo sapiens
ctgccaggctgcgcggggaggctggtcccgggctgggcaggcgggctggcctcgcgccctcgaggcacccggcggcgctggct

SEQ ID NO: 11 - DNA, 15 bp, Artificial Sequence
  Description of Artificial Sequence: Synthetic primer
ccagcccgggaccag

SEQ ID NO: 12 - DNA, 21 bp, Artificial Sequence
  Description of Artificial Sequence: Synthetic primer
gcaatagagggcaagcaaggt

SEQ ID NO: 13 - DNA, 14 bp, Artificial Sequence
  Description of Artificial Sequence: Synthetic primer
cctggcaggtctcc

SEQ ID NO: 14 - DNA, 999 bp, Homo sapiens
gcgcaggacagcactttgctcaagaccctctttaaggggtctttcacgggtcctcaagaggttgcaaagcccctgcaacaatagacaaaggagccccctacgcccgcccacttcgagcactccacgttcctcgtctcccactctggccggtcagcttcagctctggctcttgctgagcgtggagggcgcaggggcaggatccggcgcccaccgctggggcgcatgcacctcctcgcctcctcgcagggcggtggggcgcagcggttctatccccctccccgaggcggggaagaaggtctggaaagaaacgtgggttcgaggcggagaggaaaagcggacccacctgccaggctgcgcggggaggctggtcccgggctgggcaggcgggctggcctcgcgccctcgaggcacccggcggcgctggctgtgcggaggggcgccggcgcggccgtatttgtacccgcgggccctcacatggtctgatctctagatagccgccgccaaagagctcttgaagaatttttgcgtcactttgaggcgaataaacttaatgcttccccgcggccgcggctccgcgctcccgctggatggggttgcgctcgccagggaggggccgcgctacggggcggggtgcgcgcccgaccccagagccaggaggggagggacccccgacacacacacacgctcgcagggaggagcggagcgcggagcagccggcagggcagggccgagcgaggaatcctgactttcctgctctttaactttgcgggaggggggaggctgcacaaaggagcaggtgtgcgcctcccctgccgccccgcgcccacaggacggcacacaggggtctgctcgtgccgctttctcttgacctcggacacctccccatcgtccccatcccgaaagctgctctctctttcttcctgggacctagcagtactcgatacccatgcgtttatctgactgaccggctgggatgtgagctcagcttagaggcccttg

(19) United States
(12) Patent Application Publication
(10) Pub. No.: US 2007/0248576 A1
(43) Pub. Date: Oct. 25, 2007

(54) Regulation of the serotonin reuptake transporter and disease

(76) Inventors: Robert Philibert, University Heights, IA (US); Anup Madan, Bellevue, WA (US)

Correspondence Address: VIKSNINS HARRIS & PADYS PLLP, P.O. BOX 111098, ST. PAUL, MN, 55111-1098, US

(21) Appl. No.: 11/644,198

(22) Filed: Dec. 22, 2006

Related U.S. Application Data
(60) Provisional application No. 60/755,493, filed on Dec. 30, 2005.

(51) Int. Cl. C12Q 1/68; C12N 5/00

(52) U.S. Cl. 424/93.7; 435/325; 435/375; 435/6; 536/23.1

(57) ABSTRACT

Described herein is a CpG island in the 5′ region of the 5HTT gene that contains an alternative exon 1 and promoter for 5HTT. Methylation at this CpG island is associated with decreased levels of 5HTT mRNA, and this effect is evident when 5HTTLPR genotype is taken into account. Thus, this methylation status indicates 5HTT mRNA production, which serves as an indicator for the expression of the transporter and of a subject's vulnerability to diseases related to serotonergic activity. Accordingly, certain embodiments of the present invention provide diagnostic methods for determining whether a subject has, or is at risk for developing, a disease associated with serotonergic activity.

### RELATED APPLICATION(S)

This patent document claims the benefit of priority of U.S. application Ser. No. 60/755,493, filed Dec. 30, 2005, which application is herein incorporated by reference.

### STATEMENT OF GOVERNMENT SUPPORT

Work related to this patent document was funded by the U.S. government. (NIH Grants K08MH064714, R01DA015789 and R01AI05326). The government may have certain rights in this patent document.

### BACKGROUND

The serotonin reuptake transporter (5HTT) is a principal regulator of serotonergic activity, and regulation of the 5HTT is thought to be an important moderator of vulnerability to neuropsychiatric illness. In attempt to understand the basis of this regulation, several gene polymorphisms that affect 5HTT mRNA levels have been described. However, in spite of intense investigation, no clear relationship between these polymorphisms and vulnerability has been described. An understanding of the relationship is important and would provide, e.g., methods for diagnosing subjects that have, or that are at risk for developing, diseases that are related to serotonergic activity.

### SUMMARY OF CERTAIN EMBODIMENTS OF THE INVENTION

Described herein is a CpG island in the 5′ region of the 5HTT gene that contains an alternative exon 1 and a promoter for 5HTT. As described herein, the methylation status of this CpG island is associated with levels (e.g., increased or decreased levels) of 5HTT mRNA. Thus, this methylation status is indicative of 5HTT mRNA production, which serves as an indicator for the expression of the transporter and of a subject's vulnerability to diseases related to serotonergic activity.

Accordingly, certain embodiments of the present invention provide a diagnostic method for determining whether a subject has a disease associated with serotonergic activity, including determining the methylation of a serotonin reuptake transporter CpG island from a biological sample from the subject, wherein an alteration of the methylation indicates that the subject has a disease associated with serotonergic activity. An alteration in methylation may be, e.g., an increase or decrease in methylation.

Certain embodiments of the present invention provide a diagnostic method for determining whether a subject is at an elevated risk for developing a disease associated with serotonergic activity, including determining the methylation of a serotonin reuptake transporter CpG island from a biological sample from the subject, wherein an alteration of the methylation indicates that the subject is at an elevated risk for developing a disease associated with serotonergic activity.

Certain embodiments of the present invention provide a method for determining the effectiveness of a treatment for treating a disease associated with serotonergic activity, including comparing the methylation of a serotonin reuptake transporter CpG island from a biological sample from a subject that has, or is at risk for developing, a disease associated with serotonergic activity following administration of the treatment to the subject with the methylation of the serotonin reuptake transporter CpG island from a biological sample from the subject prior to administration of the treatment.

Certain embodiments of the present invention provide a method for determining whether a treatment modulates the expression of a serotonin reuptake transporter, including determining whether the treatment alters the methylation of a serotonin reuptake transporter CpG island, wherein an alteration of the methylation of the serotonin reuptake transporter CpG island indicates that the treatment modulates the expression of a serotonin reuptake transporter.

Certain embodiments of the present invention provide a method of regulating the expression of the serotonin reuptake transporter including altering the methylation of a serotonin reuptake transporter CpG island so as to regulate the expression of the serotonin reuptake transporter.

Certain embodiments of the present invention provide an isolated nucleic acid sequence including a serotonin reuptake transporter CpG island.

Certain embodiments of the present invention provide an isolated nucleic acid sequence that includes a sequence at least 80% identical to SEQ ID NO:9.

Certain embodiments of the present invention provide an isolated nucleic acid sequence that includes a sequence at least 80% identical to SEQ ID NO:10.

Certain embodiments of the present invention provide an expression cassette that includes a nucleic acid sequence of the invention.

Certain embodiments of the present invention provide a cell that includes an expression cassette of the invention.

Certain embodiments of the present invention provide a cell that includes a nucleic acid sequence of the invention.

Certain embodiments of the present invention provide a composition that includes a nucleic acid sequence of the invention.

Certain embodiments of the present invention provide a composition that includes an expression cassette of the invention.

Certain embodiments of the present invention provide a composition that includes a cell of the invention.

### BRIEF DESCRIPTION OF THE FIGURES

This patent document contains at least one drawing executed in color. Copies of this patent document with color drawings will be provided by the Office upon request and payment of the necessary fee.

FIG. 1. FIG. 1 provides an illustration of 5HTT primary gene structure as determined using the University of California, Santa Cruz (UCSC) genome browser. The location of the 14 exons identified by Lesch and colleagues (Lesch et al., J Neural Transm Gen Sect 1994; 95(2):157-162) and the new predicted gene structure is illustrated and runs right to left. The 5HTTLPR polymorphism is approximately 1400 bp upstream of exon 1 as depicted by Lesch. Chromosomal location on 17q is given as base pairs per the UCSC browser.

FIG. 2. FIG. 2 provides the sequence of the CpG island (SEQ ID NO:9) and new exon 1 (SEQ ID NO:10) as per the UCSC website. The CpG island is outlined in color with CpG residues being highlighted in red. Exon 1 (beginning at bp 427 and ending at bp 343) is underlined and runs right to left. The predicted TATA box (in the reverse strand) is double underlined. One hundred base pairs sequences flanking the island are shown in black.

FIG. 3. FIG. 3 depicts the relationship between methylation and 5HTTLPR genotype. One and two standard deviation measures are given by the bar and tip of each diamond. Group size ll=21, sl=21, ss=7.

### DETAILED DESCRIPTION

Described herein is a CpG island in the 5′ region of the 5HTT gene that contains an alternative exon 1 and a promoter for 5HTT (see Philibert et al., Am J. Med. Gen. Part B (Neuropsychiatric Genetics) 144B:101-105 (2006)). As described herein, the methylation status of this CpG island is associated with altered, e.g., decreased or increased, levels of 5HTT mRNA. Thus, this methylation status is indicative of 5HTT mRNA production, which serves as an indicator for the expression of the transporter and of a subject's vulnerability to diseases related to serotonergic activity.

Accordingly, certain embodiments of the present invention provide a diagnostic method for determining whether a subject has a disease associated with serotonergic activity, including determining the methylation of the serotonin reuptake transporter CpG island from a biological sample from the subject, wherein an alteration of the methylation (e.g., as compared to a subject that does not have such a disease) indicates that the subject has a disease associated with serotonergic activity.

Certain embodiments of the present invention provide a diagnostic method for determining whether a subject is at an elevated risk for developing a disease associated with serotonergic activity, including determining the methylation of the serotonin reuptake transporter CpG island from a biological sample from the subject, wherein an alteration of the methylation (e.g., as compared to a subject that does not have such a disease) indicates that the subject is at an elevated risk for developing a disease associated with serotonergic activity.

In some embodiments, the determination of the methylation of the serotonin reuptake transporter CpG island includes determining whether at least one the CpG motifs in the serotonin reuptake transporter CpG island is methylated. Some embodiments include determining whether the CpG motif at basepair 205, 715, 729 and/or 872 is methylated.

Certain embodiments of the present invention provide a method for determining the effectiveness of a treatment for treating a disease associated with serotonergic activity, including comparing the methylation of the serotonin reuptake transporter CpG island from a biological sample from a subject that has, or is at risk for developing, a disease associated with serotonergic activity following administration of the treatment to the subject with the methylation of the serotonin reuptake transporter CpG island from a biological sample from the subject prior to administration of the treatment. An alteration of the methylation, e.g., an increase in the methylation, can be used as an indication of whether the treatment is effective.

In some embodiments, the comparison includes comparing the methylation of at least one the CpG motifs in the serotonin reuptake transporter CpG island. Some embodiments involve comparing whether the CpG motif at basepair 205, 715, 729 and/or 872 is methylated.

Certain embodiments of the present invention provide a method for determining whether a treatment modulates the expression of a serotonin reuptake transporter, including determining whether the treatment alters the methylation of the serotonin reuptake transporter CpG island, wherein a alteration of the methylation of the serotonin reuptake transporter CpG island indicates that the treatment modulates the expression of a serotonin reuptake transporter. In some embodiments, the treatment includes the administration of a compound, such as a compound that is being evaluated for potential efficacy for treating a disease associated with serotonergic activity.

Certain embodiments of the present invention provide a method of regulating the expression of the serotonin reuptake transporter including altering the methylation of the serotonin reuptake transporter CpG island so as to regulate the expression of the serotonin reuptake transporter.

In some embodiments, the alteration of methylation occurs at one or more of the CpG motifs in the serotonin reuptake transporter CpG island. In some embodiments, the alteration of methylation occurs at one or more of the CpG motifs at basepair 205, 715, 729 and/or 872 in the serotonin reuptake transporter CpG island.

In some embodiments, the alteration occurs in vitro. In some embodiments, the alteration occurs in a subject in vivo.

In some embodiments, the subject is a mammal. In some embodiments, the subject is a male. In some embodiments, the subject is a female. In some embodiments, the subject is a human. In some embodiments, the subject has the 5HTTLPR polymorphism.

In some embodiments, the disease associated with serotonergic activity is at least one of depression, aggressive behavior, anxiety, suicide, premenstrual syndrome, an eating disorder, anorexia, bulimia, migraine, bipolar disorder, schizophrenia, alcoholism, autism, attention deficit hyperactivity disorder, or obsessive compulsive disorder. In some embodiments, the disease is depression or schizophrenia. In some embodiments, the disease is depression. In some embodiments, the disease is schizophrenia.

In some embodiments, the sample is a tissue sample. In some embodiments, the sample is blood. In some embodiments, the sample is cerebrospinal fluid.

Certain embodiments of the present invention provide an isolated nucleic acid sequence including a serotonin reuptake transporter CpG island.

Certain embodiments of the present invention provide an isolated nucleic acid sequence that includes a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:9.

Certain embodiments of the present invention provide an isolated nucleic acid sequence that includes a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:10.

In some embodiments, the nucleic acid sequence of the invention is methylated, e.g., at a CpG motif. In some embodiments, the nucleic acid sequence of the invention is methylated at one or more of positions 205, 715, 729 and/or 872 of serotonin reuptake transporter CpG island. In some embodiments, the nucleic acid sequence of the invention is not methylated.

Certain embodiments of the present invention provide an expression cassette that includes a nucleic acid sequence of the invention.

Certain embodiments of the present invention provide a cell that includes an expression cassette of the invention.

Certain embodiments of the present invention provide a cell that includes a nucleic acid sequence of the invention.

Certain embodiments of the present invention provide a composition that includes a nucleic acid sequence of the invention.

Certain embodiments of the present invention provide a composition that includes an expression cassette of the invention.

Certain embodiments of the present invention provide a composition that includes a cell of the invention.

Certain embodiments of the present invention provide compositions, e.g., pharmaceutical compositions that include a cell, expression cassette, and/or nucleic acid sequence of the invention. The composition may also include a pharmaceutically acceptable carrier.

Certain embodiments of the present invention provide a cell, expression cassette, and/or nucleic acid sequence of the invention for use in medical treatment or diagnosis.

Certain embodiments of the present invention also provide the use of a cell, expression cassette, and/or nucleic acid sequence of the invention to prepare a medicament useful for treating at least one disease that is related to serotonergic activity in an animal.

Thus, described herein is a CpG island in the 5′ region of the 5HTT gene that contains an alternative exon 1 and promoter for 5HTT. As described herein, the methylation status of this CpG island was determined. and the relationship between methylation and 5HTT mRNA levels was determined. Methylation at this CpG island is associated with decreased levels of 5HTT mRNA, but this effect is evident only when 5HTTLPR genotype is taken into account. Thus, this methylation status indicates 5HTT mRNA production, which serves as an indicator for the expression of the transporter and of a subject's vulnerability to diseases related to serotonergic activity.

Serotonin and the Serotonin Transporter

The neurotransmitter serotonin is widely distributed in the brain, and serotonergic activity plays a prominent role in the regulation of mood, cognition, and several homeostatic mechanisms such as temperature regulation, food intake, and blood pressure. Alterations in serotonergic activity are thought to result in diseases such as psychiatric and neurological diseases, including aggressive behavior, anxiety, depression, suicide, premenstrual syndrome, eating disorders (e.g., anorexia and bulimia), migraines, bipolar disorder, schizophrenia, alcoholism, autism, attention deficit hyperactivity disorder, obsessive compulsive disorder and other problems associated with impulse control. Thus, a disease related to serotonergic activity includes those listed herein and any other disease that is related, at least in part, to alterations in serotonergic activity.

The serotonin reuptake transporter (5HTT), located at 17q11, is an important regulator of serotonergic neurotransmission and is thought to be involved in vulnerability to behavioral illness. In efforts to determine whether genetic variation of 5HTT contributes variation in serotonin reuptake, a large number of studies have been conducted. To a large extent, these studies have failed to demonstrate a significant role for coding variants in regulating serotonin re-uptake. In contrast, several studies have demonstrated that non-coding variants have a significant role in regulating serotonin transport by altering 5HTT mRNA levels. The best characterized of these non-coding variants, the 5HTTLPR polymorphism, has effects on 5HTT mRNA levels in lymphoblasts, in platelet serotonin reuptake, and platelet serotonin content. Other non-coding variants, including the Long G polymorphism and an intron 2 polymorphism denoted as the STin2 variable nucleotide repeat (VNTR), may also have effects on 5HTT mRNA production, although these effects are less well established.

Given the importance of this gene in regulating serotonergic neurotransmission, at least several hundred studies have examined the role of genetic variation in 5HTT, in particular, the 5HTTLPR polymorphism, in mediating vulnerability to neuropsychiatric illness such as depression, schizophrenia and autism. The results of these studies have been mixed, with many studies inferring that the 5HTTLPR polymorphism may have effect on vulnerability to a broad range of behavioral illnesses. However, with the exception of alcoholism and schizophrenia, meta-analyses of these studies have not demonstrated a significant role for pure genetic effects of these 5HTT polymorphisms in neuropsychiatric illness.

Despite this lack of clearly identifiable pure genetic (G) effects, some studies have demonstrated that significant gene environment interactions (GxE) occur at the 5HTTLPR locus with respect to depression. However, the exact mechanism through which the environment could alter the effects of the 5HTTLPR on 5HTT function is not clear.

DNA Methylation

DNA does not exist as naked molecules in the cell. For example, DNA is associated with proteins called histones to form a complex substance known as chromatin. Chemical modifications of the DNA or the histones alter the structure of the chromatin without changing the nucleotide sequence of the DNA. Such modifications are described as “epigenetic” modifications of the DNA. Changes to

the structure of the chromatin can have a profound influence on gene expression. If the chromatin is condensed, factors involved in gene expression may not have access to the DNA, and the genes will be switched off. Conversely, if the chromatin is “open,” the genes can be switched on. Some important forms of epigenetic modification are DNA methylation and histone deacetylation. DNA methylation is a chemical modification of the DNA molecule itself and is carried out by an enzyme called DNA methyltransferase. Methylation can directly switch off gene expression by preventing transcription factors binding to promoters. A more general effect is the attraction of methyl-binding domain (MBD) proteins. These are associated with further enzymes called histone deacetylases (HDACs), which function to chemically modify histones and change chromatin structure. Chromatin-containing acetylated histones are open and accessible to transcription factors, and the genes are potentially active. Histone deacetylation causes the condensation of chromatin, making it inaccessible to transcription factors and causing the silencing of genes.

CpG islands are short stretches of DNA in which the frequency of the CpG sequence is higher than other regions. The “p” in the term CpG indicates that cysteine (“C”) and guanine (“G”) are connected by a phosphodiester bond. CpG islands are often located around promoters of housekeeping genes and many regulated genes. At these locations, the CG sequence is not methylated. By contrast, the CG sequences in inactive genes are usually methylated to suppress their expression.

About 56% of human genes and 47% of mouse genes are associated with CpG islands. Often, CpG islands overlap the promoter and extend about 1000 base pairs downstream into the transcription unit. Identification of potential CpG islands during sequence analysis helps to define the extreme 5′ ends of genes, something that is notoriously difficult with cDNA-based approaches.

The methylation of the serotonin reuptake transporter CpG island can be determined by the art worker using any method suitable to determine such methylation. For example, the art worker can use a bisulfite reaction-based method for determining such methylation.

The Serotonin Transporter, DNA Methylation and Disease

Reported herein is the discovery of a CpG island ˜11000 bp upstream of the 5HTTLPR that overlaps with an alternative first exon and an alternative promoter for 5HTT. Using 49 lymphoblast cell lines previously ascertained for mRNA levels and 5HTTLPR genotype, results presented herein indicate that the methylation status of this CpG element has a significant impact on mRNA levels. Because this methylation affects the production of the transporter, these findings provide a useful tool for determining whether a subject is at risk for developing a disease that is associated with serotonergic activity. These findings also provide a useful tool for determining whether a subject has a disease that is associated with serotonergic activity. These findings also indicate that subjects with specific genotypes have a greater risk for developing, or having, a disease that is associated with serotonergic activity and also indicate that those patients may be more amenable to treatments that affect the expression of the transporter, for example, by modulating (e.g., increasing or decreasing) the methylation. Thus, methylation at the 5HTT locus is an important regulator of serotonergic function and the extent of methylation may be dependent on 5HTTLPR genotype. Furthermore, environmental pathways likely alter methylation at this locus and, methylation of this locus appears to be an indicator of acute illness.

The primary structure of the 5HTT locus needs to be revised in view of the results presented herein to include these new upstream exon, promoter, and CpG elements. The results presented herein indicate that potential gene promoters exist in both strands. Hence, the region may be more complex than current analyses indicate. At the same time, these new data indicate that this region contains a promoter for the 5HTT gene and suggest that the 5HTTLPR should now be considered as a potential intronic rather than promoter polymorphism. Thus, certain embodiments of the present invention provide nucleic acid molecules (e.g., isolated nucleic acid molecules) that include the 5HTT locus, exon, promoter, and/or CpG element and polypeptides encoded by and/or produced by such nucleic acid molecules.

These results, which demonstrate a relationship between the 5HTTLPR genotype and methylation, may also partially reconcile prior data. In 1996, Lesch and colleagues reported a significant main effect of 5HTTLPR genotype on mRNA transcription. The main effect of 5HTTLPR genotype, which in part relied on plasmid expression assays, could not be substantiated in a later report by Hranlovich and colleagues using a larger collection of cell lines. (Hranilovic et al., Biol Psychiatry 2004; 55(11):1090-1094) But recent studies, which used a total of 84 cell lines, did confirm the earlier report, but with a smaller main effect of 5HTTLPR genotype on mRNA production. (Bradley et al., Am J Med Genet B Neuropsychiatr Genet., 136(1), 58-61 (2005)) Since some of earlier Lesch data were based on experiments using plasmid constructs that are generally not affected by methylation, the current data could explain why larger effect sizes were observed in the 1996 Lesch communication.

In summary, methylation of a CpG island associated with the 5HTT gene significantly affects mRNA transcription, and the magnitude of that effect is dependent on 5HTTLPR genotype. Furthermore, the current understanding of 5HTT gene structure needs to be modified to include the current results.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The term “nucleotide sequence” refers to a polymer of DNA or RNA which can be single- stranded or double-stranded, optionally containing synthetic, non-natural or altered nucleotide bases capable of incorporation into DNA or RNA polymers.

The terms “nucleic acid,” “nucleic acid molecule,” or “polynucleotide” are used interchangeably and may also be used interchangeably with gene, cDNA, DNA and RNA encoded by a gene.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

“Naturally occurring” is used to describe a composition that can be found in nature as distinct from being artificially produced. For example, a nucleotide sequence present in an organism , which can be isolated from a source in nature and which has not been intentionally modified by a person in the laboratory, is naturally occurring.

A “vector” includes viral vectors, as well as any plasmid, cosmid, phage or binary vector in double or single stranded linear or circular form that may or may not be self transmissible or mobilizable, and that can transform prokaryotic or eukaryotic host either by integration into the cellular genome or exist extrachromosomally (e.g., autonomous replicating plasmid with an origin of replication).

An “expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest but may also code for a functional RNA of interest, for example an antisense RNA, a nontranslated RNA in the sense or antisense direction, or a siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Functional RNA” refers to sense RNA, antisense RNA, ribozyme RNA, siRNA, or other RNA that may not be translated but yet has an effect on at least one cellular process.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters. Examples of promoters that may be used in the present invention include CMV, RSV, poIII and poIII promoters.

A “5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

A “3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

A “promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

“Constitutive expression” refers to expression using a constitutive promoter. “Conditional” and “regulated expression” refer to expression controlled by a regulated promoter.

“Operably-linked” refers to the association of nucleic acid sequences on a single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein.

The term “altered level of expression” refers to the level of expression in transgenic cells or organisms that differs from that of normal or untransformed cells or organisms.

“Antisense inhibition” refers to the production of antisense RNA transcripts capable of suppressing the expression of protein from an endogenous gene or a transgene.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a fill-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm. Non-limiting examples of such mathematical algorithms are the algorithm of Myers and Miller (Myers and Miller, CABIOS, 4, 11(1988)); the local homology algorithm of Smith et al. (Smith et al., Adv. Appl. Math., 2, 482 (1981)); the homology alignment algorithm of Needleman and Wunsch (Needleman and Wunsch, J M B, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)), are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present invention, comparison of nucleotide sequences for determination of percent sequence identity to the promoter sequences disclosed herein may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, J M B, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl Anal Biochem. 138(2):267-84 (1984); Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration is increased so that a higher temperature can be used. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. For short nucleotide sequences (e.g., about 10 to 50 nucleotides), stringent conditions typically involve salt concentrations of less than about 1.5 M, less than about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids that have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

The invention will now be illustrated by the following non-limiting example.

### EXAMPLE

The lymphoblast cell line 5HTT mRNA levels and 5HTTLPR genotype were determined (Bradley et al., Am J Med Genet B Neuropsychiatr Genet., 136(1), 58-61 (2005)). Briefly, the lymphoblast cell lines were grown using standard serum based growth media supplemented with L-glutamine and penicillin/streptomycin. One day prior to RNA extraction, this media was changed.

RNA from these lines was prepared using the Invitrogen™ (Invitrogen™, Carlsbad, Calif.) total RNA purification kit, and 5HTT mRNA levels were determined using the comparative C_{T} method using the LDHA and GAPDH as normalizing controls (Dheda et al., Biotechniques 2004; 37(1):112-4, 116, 118-9). Relative levels (Z-scores) of 5HTT mRNA levels were calculated (Fleiss, Statistical Methods for Rates and Proportions. 2nd ed. New York, N.Y.: John Wiley & Sons Inc; 1981).

Genotype at the 5HTTLPR locus was determined using PCR, electrophoresis and detection conditions as previously described (Cadoret et al., (2003) Comprehensive Psychiatry 44: 88-101; Bradley et al., (2005) Am J Med Genet B Neuropsychiatr Genet. 136: 58-61).

The existence, location, size and sequence of the CpG island were identified using the default browser settings of the University of California Genome Browser (UCSC) website (world-wide-web at genome.ucsc.edu) The existence of the alternative splicing form of 5HTT was also determined using the default Aceview settings of the browser. The identification and analysis of putative promoter sequence was conducted using the ProScan suite of programs (at thr.cit.nih.gov/molbio/proscan/) using the default settings (Prestridge, Methods Mol Biol 2000; 130:265-95) as per previous gene characterization protocols (Philibert et al., Gene 2000; 246(1-2):303-310; Philibert et al., Human Genetics 1999; 105(1-2):174-178). All sequences are freely available from the website.

After extraction from the lymphoblast cell lines using high salt extraction, the DNA for these studies was further purified using phenol/chloroform extraction. Five μg aliquots of DNA from each sample then underwent bisulfite modification using a Methyl Easy™ kit from Human Genetic Signatures (Macquarie Park, Australia) according to the manufacturer's instructions.

For ease of amplification, the 799 bp CpG island was broken into two overlapping contigs totaling 882 bp. Nested sets of primers in non CpG rich flanking regions that selectively recognize only the bisulfite modified DNA segments were designed to amplify the region. These nested primers and a HotStar Taq® (Qiagen, Valencia, Calif.) kit were used in combination with a touchdown PCR protocol to amplify aliquots of bisulfite treated DNA in a two stage process. The first contig was amplified using the primer pair (Forward) TAAGGGTTTTTAAGTTGAGTTTATATTTTAGT (SEQ ID NO:1) and (Reverse) CTAATCCCRAACTAAACAAACRAACTAA (SEQ ID NO:2) in the first round, and then the primer pair (Forward) GTATGGGTATYGAGTATTGTTAGGTTTT (SEQ ID NO:3) and (Reverse) AACCCTCACATAATCTAATCTCTAAATAA (SEQ ID NO:4) in the second round of amplification. The second contig was amplified using the primer pair (Forward) CACTTTACTCAAAACCCTCTTTAAAAAA (SEQ ID NO:5) and (Reverse) GTTTTTTTTTGGYGAGYGTAATTTTATTT (SEQ ID NO:6) and then the primer pair F-AAACCCCTACAACAATAAACAAAAAAA (SEQ ID NO:7) and (Reverse) GGGGAAGTATTAAGTTTATTCGTTTT (SEQ ID NO:8) in the second round of PCR. The PCR fragments resulting from the second round of amplification of both contigs, which were 585 bp and 493 bp, respectively, were then subcloned using a TOPO® OD cloning kit according to manufacturer's suggestions (Invitrogen, Carlsbad, Calif.).

An average of 10 clones were sequenced from each sample (approximately 1,000 total sequence traces) using standard florescent sequencing techniques and an ABI 3730 capillary sequencing machine (Applied Biosystems, Foster City, Calif.). The presence and absence of methylation was tabulated for each residue and for the segment as a whole. Sequence data was analyzed using programs phred-Phrap (Ewing, B. and Green, P., Genome Res, 1998. 8(3): p. 186-94, world-wide-web at phrap.org/phredphrapconsed.html) and viewed using consed (Gordon et al., Genome Res, 1998. 8(3): p. 195-202; Gordon D: Viewing and Editing Assembled Sequences Using Consed. In Current Protocols in Bioinformatics (A D Baxevanis and D B Davison, eds). Section 11.2.1-11.2.43 (2004)). The extent of methylation at each CpG site was quantitated using bioinformatics tools. All software tools were written in Perl 5, a programming language which is available for all common computer platforms. The input format for this program is a single file containing the mother-sequence followed by the bisulfite generated sequences in FASTA format (>name[new-line]-sequence[new-line]). The program compares the mother-sequence with the bisulfite generated sequences below in the file “filename”’, and generates individual files carrying the FASTA-name extended by “.seq1”. These files follow the FASTA-standard but cytosines in the bisulfite sequences are converted into uppercase “C”, and thymines that used to be cytosines in the mother-sequence to lowercase “c”. All other bases are written in lowercase letters. From this file, a table is written into a file “fraction_methylated.txt”. The first column contains the length of the sequence analyzed, the second the patient number, the next columns contain the average methylation at cytosine sites (in percent) per position. In the last column, it calculates the average methylation for each patient. This file can be imported into conventional data representation programs. This table serves as input to draw_bisulfite.pl program that generates a graphical view in which a hollow square represents unmethylated CpG and, depending upon the fraction methylation at each CpG site, it fills the square with an increasing gray scale.

Methylation and genotype data was analyzed using the JMP suite of programs using two tailed univariate and bivariate analyses as indicated (Version 5.1, SAS Institute, Cary, S.C.).

Results

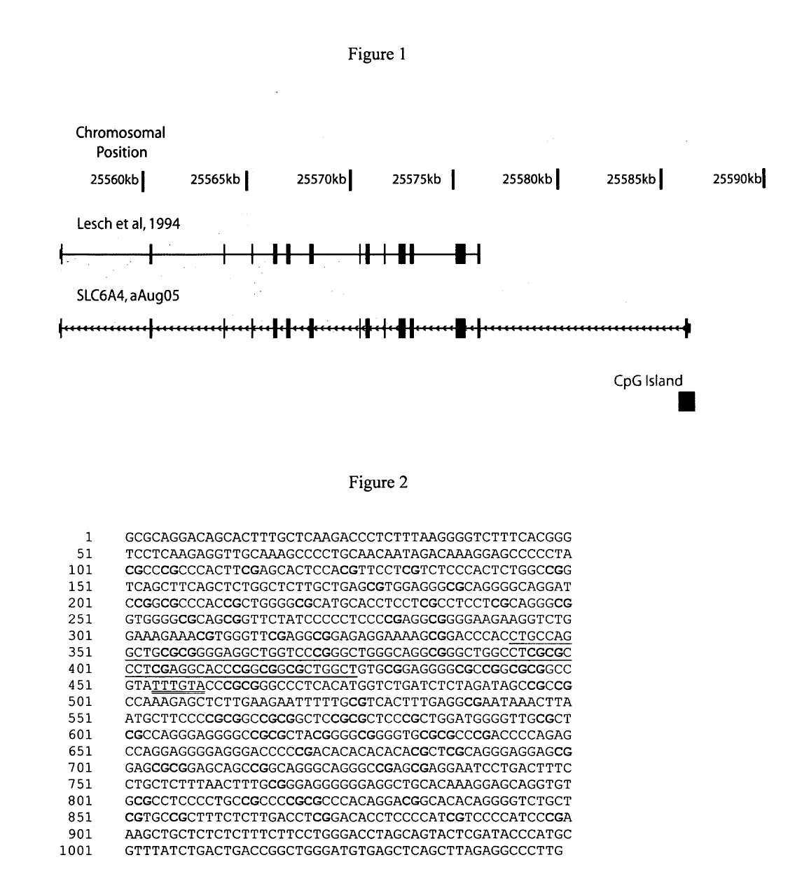

The relationship of the alternative splicing from of 5HTT and the CpG island to the more conventional view of the 5HTT gene is given in FIG. 1. The figure is adapted from the browser and illustrates that the alternative exon, which corresponds to Exon 1A (as per Mortensen et al., Brain Res. Mol. Brain Res. 68:141-149 (1999)), and CpG island are approximately 11,000 bp upstream of the exon 1B of 5HTT (Lesch et al., J Neural Transm Gen Sect 1994; 95(2):157-162).

The sequence of the putative exon one of the alternative splice form and the CpG island are given in FIG. 2. The CpG island (in color) (SEQ ID NO:9) is 799 bp and has 81 CpG residues (20.3% CpG residues). Exon 1A (FIG. 2; bp 344-428, underlined) (SEQ ID NO:10) identified is non-coding and is wholly contained inside the CpG island. Notation from the website indicates that the first exon of the putative mRNA construct (SLC6A4.aAug05) is supported fully by Genbank clone AU138385 and partially by Genbank clone BX399758.

The existence of this putative mRNA was examined using RTPCT in RNA isolated from 94 independent lymphoblast cell lines using a primer probe set whose probe straddles the exon 1 and 2 boundary (see FIG. 2). The Pearson's correlation coefficient of the Ct counts obtained using the standard (Hs00169010; n=2) and the new primer probe sets (n=4) was only 0.80 which suggests that a strict stochiometric relationship between the two sites of the recognized by the probe sets may not exist. Furthermore, strong mRNA signal was observed in all 94 lines using the new primer probe set. It should be noted that several attempts (n=4) were made to detect signal in those latter two cells lines using the new primer probe set, and that confirmation of cDNA quality for those cell lines was made using spectrophotometric and both LDHA and GAPDH internal controls.

The sequence in FIG. 1 was analyzed using Promoter Scan Version 1.7 using standard gene characterization protocols (Philibert et al., Gene 2000; 246(1-2):303-310; Philibert et al., Human Genetics 1999; 105(1-2):174-178). The analyses indicated that promoter regions are predicted on forward strand in 489 to 739 (Promoter Score 70.50: promoter cutoff=53.00) and from reverse strand in 691 to 441 (Promoter Score: 81.69). The predicted promoter in the reverse strand has a predicted transcription start site at the start site of Genbank clone AU138385 and a predicted TATA box 33 bp upstream, which suggests it may be an alternative promoter for 5HTT.

The amount of methylation at this CpG island in a cohort lymphoblast cell lines was measured. The mRNA content and 5HTTLPR genotype were previously determined. DNA from a total of 84 lines were studied, but adequate clone coverage for both ends of the CpG island were only obtained for 49 of these lymphoblast lines.

Analysis of the methylation data was surprising. Overall, there was not a main effect of overall methylation on lymphoblast 5HTT mRNA levels (p<0.18). However, before correction for multiple comparisons, the amount of methylation of 4 CpG residues, nucleotides 205, 715, 729 and 872 (see FIG. 2), were significantly associated with 5HTT mRNA levels, and there was a trend for several other CpG residues to be associated with mRNA levels as well.

Since the 5HTTLPR genotype significantly influences mRNA expression, the 5HTTLPR genotype was then controlled for and the data was reanalyzed. Using this model, the relationship between average methylation and mRNA levels was significant (p<0.01).

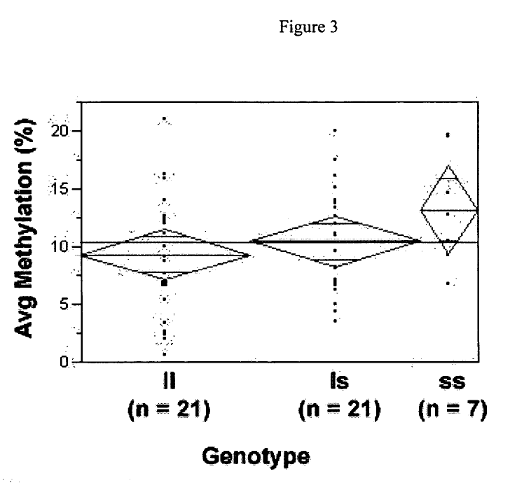

Seeking to better understand this relationship, the relationship between methylation and 5HTTLPR genotype was then analyzed. Regression analysis of the data using a model that specified the heterozygotes as intermediates demonstrated a trend for an association between genotype and methylation (p<0.10; FIG. 3). The difference between the ss (n=7) and ll (n=21) groups by themselves was not significant (p<0.11).

Additionally, a real time PCR assay has been designed and performed that recognized the new exon 1. The sequences of the primers used to amplify the sequence are (Forward) CCAGCCCGGGACCAG (SEQ ID NO:11) and (reverse) GCAATAGAGGGCAAGCAAGGT (SEQ ID NO:12). The probe sequence is 6-Fam CCTGGCAGGTCTCC (MGB NFQ) (SEQ ID NO:13). 6-Fam is the fluorescent tag, MGB stands for minor groove binder, and NFQ stands for non-fluorescent quencher. The cycling conditions used were 95° C. for 10 minutes, then 40 cycles of 95° C. for 15 sec, and 60° C. for 1 min using Taqman® Universal PCR master mix. The transcript was found in cDNA prepared from all 100 lymphoblast cell lines that were examined.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A diagnostic method for determining whether a subject has a disease associated with serotonergic activity or is at an elevated risk for developing a disease associated with serotonergic activity, comprising determining the methylation of a serotonin reuptake transporter CpG island from a biological sample from the subject, wherein an alteration of the methylation indicates that the subject has a disease associated with serotonergic activity or is at an elevated risk for developing a disease associated with serotonergic activity.

2. The method of claim 1, wherein the determination of the methylation of the serotonin reuptake transporter CpG island comprises determining whether at least one CpG motif in the serotonin reuptake transporter CpG island is methylated.

3. The method of claim 2, comprising determining whether the CpG motif at basepair 205, 715, 729 and/or 872 is methylated.

4. The method of claim 1, wherein the subject is a mammal.

5. The method of claim 4, wherein the subject is a human.

6. The method of claim 5, wherein the subject has the 5HTTLPR polymorphism.

7. The method of claim 1, wherein the disease associated with serotonergic activity is at least one of depression, aggressive behavior, anxiety, suicide, premenstrual syndrome, an eating disorder, anorexia, bulimia, migraine, bipolar disorder, schizophrenia, alcoholism, autism, attention deficit hyperactivity disorder, or obsessive compulsive disorder.

8. The method of claim 7, wherein the disease is depression or schizophrenia.

9. The method of claim 8, wherein the disease is depression.

10. The method of claim 8, wherein the disease is schizophrenia.

11. The method of claim 1, wherein the sample is a tissue sample.

12. The method of claim 1, wherein the sample is blood.

13. The method of claim 1, wherein the sample is cerebrospinal fluid.

14. A method for determining the effectiveness of a treatment for treating a disease associated with serotonergic activity, comprising comparing the methylation of a serotonin reuptake transporter CpG island from a biological sample from a subject that has, or is at risk for developing, a disease associated with serotonergic activity following administration of the treatment to the subject with the methylation of the serotonin reuptake transporter CpG island from a biological sample from the subject prior to administration of the treatment.

15. The method of claim 14, wherein the comparison comprises comparing the methylation of at least one CpG motif in the serotonin reuptake transporter CpG island.

16. The method of claim 15, comprising comparing whether the CpG motif at basepair 205, 715, 729 and/or 872 is methylated.

17. A method for determining whether a treatment modulates expression of a serotonin reuptake transporter, comprising determining whether the treatment alters the methylation of a serotonin reuptake transporter CpG island, wherein an alteration of the methylation of the serotonin reuptake transporter CpG island indicates that the treatment modulates expression of the serotonin reuptake transporter.

18. The method of claim 17, wherein the treatment comprises the administration of at least one compound.

19. A method of regulating the expression of the serotonin reuptake transporter comprising altering the methylation of a serotonin reuptake transporter CpG island so as to regulate the expression of the serotonin reuptake transporter.

20. The method of claim 19, wherein the alteration of methylation occurs at one or more CpG motif in the serotonin reuptake transporter CpG island.

21. The method of claim 20, wherein the alteration of methylation occurs at one or more of the CpG motifs at basepair 205, 715, 729 and/or 872 in the serotonin reuptake transporter CpG island.

22. The method of claim 19, wherein the alteration occurs in vitro.

23. The method of claim 19, wherein the alteration occurs in a subject in vivo.

24. An isolated nucleic acid sequence comprising a serotonin reuptake transporter CpG island.

25. An isolated nucleic acid sequence comprising a sequence at least 80% identical to SEQ ID NO:9 or SEQ ID NO:10.

26. The nucleic acid sequence of claim 25 that is methylated.

27. The nucleic acid sequence of claim 26 that is methylated at one or more of positions 205, 715, 729 and/or 872 of the serotonin reuptake transporter CpG island.

28. The nucleic acid sequence of claim 25 that is not methylated.

29. An expression cassette comprising the nucleic acid sequence of claim 25.

30. A cell comprising the expression cassette of claim 29.

31. A composition comprising the cell of claim 30.

32. A composition comprising the expression cassette of claim 29.

33. A composition comprising the nucleic acid sequence of claim 25.

34. A cell comprising the nucleic acid sequence of claim 25.

35. A composition comprising the cell of claim 34.
